SEQUENCE LISTING (4 sequences)

SEQ ID NO: 1 - protein, 667 aa, synthetic construct
QVQLVQSGAEVKKPGSSVKVSCKASGGTFRRYSISWVRQAPGQGLEWMGGIIPVFGAAKYAQKFQGRVTITADEFTSTAYMELSSLTSEDTAVYYCALSGDSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGDTGSPFVEMYSEIPEIIHMTEGSELVIPCRVTSPDITVTLKKFPLDTLIPDGKRIIWDSRKGFIISDATYKEIGLLTCEATVNGHLYKTNYLTHRQTNTIIDVVLSPSHGIELSVGEKLVLDCTARTELNVGIDFNWEYPSSKHQHKKLVNRDLKTQSGSEMKKFLSTLTIDGVTRSDQGLYTCAASSGLMTKKDSTFVRVHEK

SEQ ID NO: 2 - protein, 217 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCATWDLSLNAWVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - DNA, 2001 bp, synthetic construct
  source 1..2001: mol_type = other DNA
caggtgcagctggtgcagtccggcgccgaggtgaagaagcctggctcctccgtgaaggtgagctgtaaggcttccggcggcaccttcaggaggtacagcatcagctgggtgaggcaggcccctggccagggactggagtggatgggcggcatcatccctgtgttcggcgctgctaagtacgcccagaagttccagggccgggtgaccatcaccgccgatgagttcaccagcaccgcctacatggagctgtcctccctgacctccgaggataccgctgtgtattattgtgccctgtccggcgacagcgatgccttcgacatctggggccagggcacaatggttaccgtgtcctccgcttccaccaagggcccctccgtgttccccctggccccttcttccaagtccaccagcggcggcaccgccgctctgggatgtctggtgaaggattacttccctgagcctgtgaccgtgagctggaatagcggcgctctgaccagcggcgtgcacaccttccctgctgtgctgcagagcagcggcctgtactccctgtcctccgtggtgaccgtgcccagctcctccctgggcacccagacctacatctgtaatgtgaatcacaagcccagcaataccaaggtggacaagaaggtggagcccaagagctgcgataagacccacacctgtcctccttgtcccgcccccgagctgctgggaggaccatctgtgttcctgttccctcccaagcctaaggataccctgatgatctccaggacccctgaggtgacctgtgtggtggtggatgtgagccacgaggaccccgaggtgaagttcaactggtacgtggacggcgtggaggtgcacaatgccaagaccaagcccagggaggagcagtacgcttccacctacagggtggtgtccgtgctgaccgtgctgcaccaggactggctgaatggcaaggagtataagtgcgctgtgagcaataaggctctgcccgcccccatcgagaaaactattagtaaggccaagggccagcccagggagccccaggtgtataccctgcccccttcccgggaggagatgaccaagaaccaggtgtccctgacctgtctggtgaaaggcttctacccttccgacatcgctgtggagtgggagagcaacggccagcccgagaacaattataagaccacccctcccgtgctggacagcgatggctccttcttcctgtacagcaagctgaccgtggacaagtccaggtggcagcagggcaatgtgttcagctgctccgtgatgcacgaggctctgcacaaccactacacccagaagagcctgagcctgtcccccggcggcggaggaggatctggaggaggaggcagcggcggcggaggttctggagacaccggctcccccttcgtggagatgtactccgagatccctgagatcatccacatgaccgagggctccgagctggtgatcccctgtcgggtgaccagccccgatatcaccgtgaccctgaagaagttccctctggataccctgatccccgacggcaagaggatcatctgggatagcaggaagggcttcatcatctccgatgctacctataaggagatcggcctgctgacctgtgaggctaccgtgaatggccacctgtacaagaccaactacctgacccaccggcagaccaataccatcatcgacgtggtgctgagccctagccacggcatcgagctgtccgtgggcgagaagctggtgctggactgcaccgccaggaccgagctgaatgtgggcatcgacttcaactgggagtaccctagcagcaagcaccagcacaagaagctggtgaatagggacctgaaaactcaatctggcagcgagatgaagaagttcctgagcaccctgaccatcgatggcgtgaccaggtccgatcagggcctgtacacctgtgctgcttcttccggcctgatgaccaagaaggactccaccttcgtgagggtgcacgagaag

SEQ ID NO: 4 - DNA, 651 bp, synthetic construct
  source 1..651: mol_type = other DNA
cagagcgtgctgacccagcccccttccgctagcggcacccctggacagagggtgaccatcagctgttccggcagcagcagcaacatcggctccaacaccgtgaactggtaccagcagctgcctggcaccgcccccaagctgctgatctatagcaacaaccagcggccctccggcgtgcctgatcggttctccggctccaagtccggcacctccgcctccctggccatctccggtctgcagagcgaggatgaggccgactactactgcgctacctgggacctgagcctgaacgcttgggtggtgttcggcggcggcaccaagctgaccgtgctgggacagcctaaggctgctccctccgtgaccctgttccctcctagctccgaggagctgcaggctaataaggctaccctggtgtgcctgatctccgacttctatcccggcgccgtgaccgtggcttggaaggctgactccagccccgtgaaggccggagtggagaccaccaccccttccaagcagagcaacaataagtacgctgccagcagctatctgagcctgacccccgagcagtggaagagccaccggagctatagctgccaggtgacccacgagggctccaccgtggagaaaactgttgctcccaccgagtgtagc

(19) United States
(12) Patent Application Publication
(10) Pub. No.: US 2025/0075172 A1
(43) Pub. Date: Mar. 6, 2025

(54) PRODUCTION METHOD FOR BISPECIFIC ANTIBODY

(71) Applicant: TASLY BIOPHARMACEUTICALS CO., LTD., Shanghai (CN)

(72) Inventors: Pengyin WANG, Shanghai (CN); Wei ZHANG, Shanghai (CN); Huaibin MU, Shanghai (CN); Xiao XU, Shanghai (CN); Wei JING; Wenlei LI, Shanghai (CN); Xiaodan CAO, Shanghai (CN); Lihua XU, Shanghai (CN); Fang TIAN, Shanghai (CN); Jing LI, Shanghai (CN); Ruijing HUANG, Shanghai (CN)

(21) Appl. No.: 18/723,575

(22) PCT Filed: Dec. 20, 2022
(86) PCT No.: PCT/CN2022/140466

§ 371 Date: Jun. 24, 2024

(30) Foreign Application Priority Data
Dec. 24, 2021 (CN) 202111602161.4

(51) Int. Cl. C12N 5/00; C07K 1/18; C07K 1/22; C07K 16/22; C07K 16/28; C07K 16/46

(52) U.S. Cl. CPC C12N 5/0018; C07K 1/18; C07K 1/22; C07K 16/22; C07K 16/2827; C07K 16/468; C07K 2317/31; C12N 2500/34

(57) ABSTRACT

A production method for a bispecific antibody, comprising constructing cells for producing a bispecific antibody, screening the cells, culturing the cells obtained in step one to obtain a culture solution, and performing separation and purification to obtain the bispecific antibody. The cells comprise CHO cells. The culture mode comprises fed-batch culture or perfusion culture. Various influence factors in fermentation culture and separation and purification are comprehensively analyzed, and by effective control, all factors can effectively cooperate, so that a high-purity bispecific antibody is efficiently produced, the daily output can reach 3 g/L or above, and the purity of the purified bispecific antibody can reach 90% or above. Moreover, the production process is amplified and verified, the process is stable and reliable, the cost is low, a great breakthrough is achieved compared with an existing production method. The present application has great significance for wide clinical application of bispecific antibodies.

### CROSS-REFERENCE TO RELATED APPLICATIONS

This application is a national phase under 35 U.S.C. § 371 of International Application No. PCT/CN2022/140466 filed on Dec. 20, 2022, which claims the benefit of priority from Chinese Patent Application No. 202111602161.4 filed on Dec. 24, 2021. The entire contents of these applications is incorporated herein by reference in their entirety

### TECHNICAL FIELD

The present application belongs to the field of biotechnology, and relates to a production method for a bispecific antibody.

### BACKGROUND

Tumors may be classified into benign tumors and malignant tumors according to cell characteristics of neoplasms and degrees of harm to organisms. Malignant tumor diseases are major diseases that do harm to human health in today's society and have the second-highest degree of lethality. Common tumors include liver cancer, lung cancer, gastric cancer, breast cancer and bladder cancer.

Comprehensive treatment is generally performed on most patients due to individual differences of the malignant tumors, that is, means such as surgery, chemotherapy, radiotherapy, immunotherapy, traditional Chinese medicine treatment, interventional therapy and microwave therapy are comprehensively used to improve cure rates significantly and improve the patients' quality of life. The immunotherapy refers to a treatment method for artificially enhancing or inhibiting an immune function of an organism to achieve an object of treating a disease in a low or hyperactive immune state of the organism. Many methods of the immunotherapy are applicable to the treatment of multiple diseases. The immunotherapy is a treatment method that aims to activate a human immune system to depend on an immune function of the human immune system to kill cancer cells and tumor tissues so that tumors are controlled and removed. Different from previous surgery, chemotherapy, radiotherapy and targeted therapy, a target targeted by the immunotherapy is a human immune system instead of tumor cells and tissues. The immunotherapy includes a monoclonal antibody immune checkpoint inhibitor, a therapeutic antibody, a cancer vaccine, cell therapy and a small molecule inhibitor.

At present, antibody drugs sold on the market are mostly monoclonal antibodies. Therapeutic monoclonal antibodies have been used for treating cancers, autoimmune diseases, inflammations and other diseases. Most of the therapeutic monoclonal antibodies are specific for one target. However, drug resistance or no response may occur when monoclonal antibody therapy is performed on patients, and some diseases have influencing factors in multiple aspects in vivo. The influencing factors include different signaling pathways, different cytokines and regulatory mechanisms of receptors. Single-target immunotherapy seems not to be enough to destroy cancer cells. Therefore, to destroy the cancer cells, different drugs need to be combined, or a multi-targeting strategy of a multi-specific antibody needs to be used. For example, CN109942712A provides an anti-PD-L1/VEGF bispecific antibody. The anti-PD-L1/VEGF bispecific antibody includes an anti-PD-L1 antibody or element and an anti-VEGF antibody or element ligated to the anti-PD-L1 antibody or element so that the anti-PD-L1/VEGF bispecific antibody can bind to VEGF and PD-L1 at the same time, thereby exerting a therapeutic effect on VEGF-positive tumor cells and PD-L1-positive tumor cells. Although a bifunctional antibody is a direction of the development of an antibody drug, the bifunctional antibody faces many challenges such as a preclinical evaluation model, a low expression amount, poor stability, a complex process and a large difference in quality control. Therefore, the development of the bifunctional antibody is always beset with difficulties.

To conclude, for the problem that the clinical application of the bispecific antibody is limited by the complex industrial production and high production cost of the bispecific antibody, a method that can improve the expression amount of the bispecific antibody without affecting the safety, specificity and purity of the bispecific antibody and can reduce a production cost is urgently needed.

### SUMMARY

The present application provides a production method for a bispecific antibody. A high-purity bispecific antibody can be efficiently produced through the production method. The process is stable, reliable and low-cost, thereby significantly promoting the clinical application of the bispecific antibody.

The present application provides a production method for a bispecific antibody. The method includes the following steps:
(1) constructing cells for producing the bispecific antibody and screening; and
(2) culturing cells obtained through the screening in step (1) to obtain a culture solution, and separating and purifying to obtain the bispecific antibody;
   wherein the cells include mammalian cells; and
   a manner of the culture includes fed-batch culture or perfusion culture, wherein a medium for the fed-batch culture includes a basal medium and a feed medium, where the basal medium includes a Dynamis™ AGT™ Medium, the feed medium includes Cell Boost™ 7a and Cell Boost™ 7b, a temperature of the fed-batch culture is 31° C.-37° C., including, but not limited to, 32° C., 33° C., 34° C., 35° C. or 36° C., a pH of the fed-batch culture is 6.8-7.3, including, but not limited to, 6.9, 7.0, 7.1 or 7.2, and dissolved oxygen of the fed-batch culture is more than 10%; a medium for the perfusion culture includes a basal medium and a feed medium, wherein the basal medium includes an Eden-300S Medium and a High-Intensity Perfusion CHO Medium, a temperature of the perfusion culture is 31° C.-37° C., including, but not limited to, 32° C., 33° C., 34° C., 35° C. or 36° C., a pH of the fed-batch culture is 6.8-7.3, including, but not limited to, 6.9, 7.0, 7.1 or 7.2, and dissolved oxygen of the perfusion culture is more than 10%.

In the present application, a strain cell that can produce the bispecific antibody at a high yield is screened out, and the high-yielding strain cell is cultured. Multiple factors affecting fermentation culture are comprehensively analyzed, a fermentation manner, a medium combination, a culture temperature, a culture pH and dissolved oxygen are controlled, and all the factors are cooperated, thereby efficiently producing the bispecific antibody.

Preferably, the bispecific antibody includes a PD-L1/VEGF bispecific antibody.

Preferably, an amino acid sequence of the PD-L1/VEGF bispecific antibody includes sequences shown in SEQ ID NO. 1 and SEQ ID NO. 2.

(a heavy chain of the bispecific antibody):
SEQ ID NO. 1
QVQLVQSGAEVKKPGSSVKVSCKASGGTFRRYSISWVRQAPGQGLEWMGG
IIPVFGAAKYAQKFQGRVTITADEFTSTAYMELSSLTSEDTAVYYCALSG
DSDAFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCAVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGGGG
GSGGGGSGGGGSGDTGSPFVEMYSEIPEIIHMTEGSELVIPCRVTSPDIT
VTLKKFPLDTLIPDGKRIIWDSRKGFIISDATYKEIGLLTCEATVNGHLY
KTNYLTHRQTNTIIDVVLSPSHGIELSVGEKLVLDCTARTELNVGIDFNW
EYPSSKHQHKKLVNRDLKTQSGSEMKKFLSTLTIDGVTRSDQGLYTCAAS
SGLMTKKDSTFVRVHEK.
(a light chain of the bispecific antibody):
SEQ ID NO. 2
QSVLTQPPSASGTPGQRVTISCSGSSSNIGSNTVNWYQQLPGTAPKLLIY
SNNQRPSGVPDRFSGSKSGTSASLAISGLQSEDEADYYCATWDLSLNAWV
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS.

Preferably, the mammalian cells include HEK 293 cells or Chinese hamster ovary (CHO) cells, preferably the Chinese hamster ovary cells.

In the present application, preparing the medium into the Dynamis™ AGT™ Medium can further improve a yield of the bispecific antibody.

Preferably, the basal medium for the fed-batch culture contains Pluronic® F-68 BioChemica. In the present application, adding Pluronic® F-68 BioChemica to the medium can effectively solve the problem of cell agglomeration in a culture process, which is conducive to the rapid growth of the cells.

Preferably, a fed-batch proportion of Cell Boost™ 7a is 2% to 3%, including, but not limited to, 2.2%, 2.4%, 2.6%, 2.7%, 2.8% or 2.9%, and a fed-batch proportion of Cell Boost™ 7b is 0.2% to 2.5%, including, but not limited to, 0.3%, 0.4%, 0.6%, 1%, 1.2%, 1.5%, 1.8%, 2%, 2.2%, 2.3% or 2.4%.

Preferably, a seeding density of the fed-batch culture is not less than 0.15×10⁶ cells/mL, including, but not limited to, 0.36×10⁶ cells/mL, 0.38×10⁶ cells/mL, 0.4×10⁶ cells/mL, 0.45×10⁶ cells/mL, 0.5×10⁶ cells/mL or 0.6×10⁶ cells/mL.

Preferably, the fed-batch further includes adding glucose.

Preferably, a fed-batch amount of glucose is 1.0-10.0 g/L, including, but not limited to, 1 g/L, 2 g/L, 3 g/L, 4 g/L, 5 g/L, 6 g/L, 7 g/L, 8 g/L, 9 g/L or 10 g/L.

Preferably, the feed medium for the perfusion culture includes Eden-F400a and Eden-F200b.

Preferably, the separation and the purification in step (2) include the following steps:
(1′) performing depth filtration on the culture solution to obtain a clear solution;
(2′) performing affinity chromatography on the clear solution;
(3′) adjusting a pH of a product obtained through the affinity chromatography, and incubating the product;
(4′) adjusting a pH of a product obtained through the incubation, and performing depth filtration on the product;
(5′) performing anion-exchange chromatography on a product obtained through the depth filtration;
(6′) performing cation-exchange chromatography on a product obtained through the anion-exchange chromatography; and
(7′) performing nanofiltration on a product obtained through the cation-exchange chromatography.

In the present application, the bispecific antibody has a certain proportion of multimers, which seriously affects a yield and protein purity in a purification process. Improving a separation degree between the multimers and the bispecific antibody by controlling a purification process can further improve the yield and purity of the bispecific antibody.

Preferably, a filter element of a filter for the depth filtration in step (1′) includes a Zeta Plus EZP filter element E16E07A60SP02A (3M Company).

Preferably, an elution buffer for the affinity chromatography in step (2′) includes acetic acid and sodium acetate.

Preferably, a filler of a chromatography column for the affinity chromatography in step (2′) includes MabSelect PrismA.

Preferably, the pH in step (3′) is 3-4.

Preferably, in step (3′), the incubation is performed at a temperature of 18-26° C., including, but not limited to, 19° C., 20° C., 21° C., 22° C., 23° C., 24° C. or 25° C., and the incubation is performed for 50-70 min, including, but not limited to, 51 min, 52 min, 53 min, 54 min, 55 min, 56 min, 60 min, 61 min, 62 min, 65 min, 66 min, 67 min, 68 min or 69 min.

Preferably, a filler of a chromatography column for the anion-exchange chromatography in step (5′) includes Capto adhere.

Preferably, a load of Capto adhere is set to ≤30 g/L.

Preferably, the anion-exchange chromatography in step (5′) is performed at a pH of 5.8-6.0, preferably 5.9.

Preferably, a filler of a chromatography column for the anion-exchange chromatography in step (6′) includes Ceramic CM and/or Nuvia HR-S.

Preferably, an equilibration buffer for the cation-exchange chromatography in step (6′) includes acetic acid and sodium acetate.

Preferably, the cation-exchange chromatography in step (6′) is performed at a pH of 5.4-5.6, preferably 5.5.

Preferably, an eluent for the cation-exchange chromatography in step (6′) includes arginine.

Preferably, a concentration of arginine in the eluent is 0.18-0.20 mol/L.

Preferably, the separating and purifying further include steps of preparing an antibody stock solution.

Preferably, a preparation method for the antibody stock solution includes:
performing ultrafiltration on the product obtained through the cation-exchange chromatography, and performing filtration on a product obtained through the ultrafiltration by using a sterile filtration membrane to obtain the antibody stock solution.

As a preferred technical solution, the production method for a bispecific antibody includes the following steps:
(1) constructing cells for producing the bispecific antibody, and screening;
(2) culturing cells obtained through the screening in step (1) to obtain a culture solution;
(3) performing depth filtration on the culture solution by using a filter with a Zeta Plus EZP filter element E16E07A60SP02A to obtain a clear solution;
(4) filling a chromatography column with MabSelect PrismA, performing affinity chromatography on the clear solution, and eluting the clear solution with an elution buffer containing acetic acid and sodium acetate;
(5) adjusting a pH of a product obtained through the affinity chromatography to 3.5±0.1, and incubating the product for 50-70 min at 18° C.-26° C.;
(6) adjusting a pH of a product obtained through the incubation to 5.4-5.6, and performing depth filtration on the product;
(7) filling a chromatography column with Capto adhere, equilibrating the column with an equilibration buffer containing acetic acid and sodium acetate, and performing anion-exchange chromatography on a product obtained through the depth filtration;
(8) filling a chromatography column with Ceramic CM and/or Nuvia HR-S, equilibrating the column with an equilibration buffer containing acetic acid and sodium acetate, and performing cation-exchange chromatography on a product obtained through the anion-exchange chromatography;
(9) performing nanofiltration on a product obtained through the cation-exchange chromatography; and
(10) performing ultrafiltration on a product obtained through the nanofiltration, and performing filtration on a product obtained through the ultrafiltration by using a sterile filtration membrane to obtain the antibody stock solution.

A manner of the culture includes fed-batch culture or perfusion culture.

A medium for the fed-batch culture includes a basal medium and a feed medium, wherein the basal medium includes a Dynamis™ AGT™ Medium, the feed medium includes Cell Boost™ 7a and Cell Boost™ 7b, a temperature of the fed-batch culture is 31° C.-37° C., a pH of the fed-batch culture is 6.8-7.3, and dissolved oxygen of the fed-batch culture is more than 10%.

A medium for the perfusion culture includes a basal medium and a feed medium, where the basal medium includes an Eden-300S Medium and a High-Intensity Perfusion CHO Medium, a temperature of the perfusion culture is 31° C.-37° C., a pH of the perfusion culture is 6.8-7.3, and dissolved oxygen of the perfusion culture is more than 10%.

Compared with the prior art, the present application has the beneficial effects described below. In the present application, various influencing factors in the fermentation culture, the separation and the purification are comprehensively analyzed. Through effective control, all the factors can be effectively cooperated, thereby efficiently producing the high-purity bispecific antibody whose daily yield can reach more than 3 g/L. After the purification, the SEC-HPLC purity of the bispecific antibody can reach more than 90%. Moreover, the production process is amplified and verified. The process is stable, reliable and low-cost, and compared with an existing production method, a great breakthrough is achieved, which is of great significance for the widespread clinical application of the bispecific antibody.

### BRIEF DESCRIPTION OF DRAWINGS

FIG. 1 is a structure diagram of a B1962-vector-3-pCHUGUN-Kan plasmid.

FIG. 2 is a graph illustrating growth curves of cells in 5 L reactors.

FIG. 3 is a graph illustrating metabolism curves of glucose in 5 L reactors.

FIG. 4 is a graph illustrating metabolism curves of lactic acid in 5 L reactors.

FIG. 5 is a graph illustrating metabolism curves of ammonium in 5 L reactors.

FIG. 6 is a graph illustrating expression yields of proteins in 5 L reactors.

FIG. 7 is a graph illustrating growth curves of cells in 200 L reactors.

FIG. 8 is a graph illustrating metabolism curves of glucose in 200 L reactors.

FIG. 9 is a graph illustrating metabolism curves of lactic acid in 200 L reactors.

FIG. 10 is a graph illustrating expression yields of proteins in 200 L reactors.

FIG. 11 is a graph illustrating densities of perfusion cultured cells.

FIG. 12 is a graph illustrating viability rates of perfusion cultured cells.

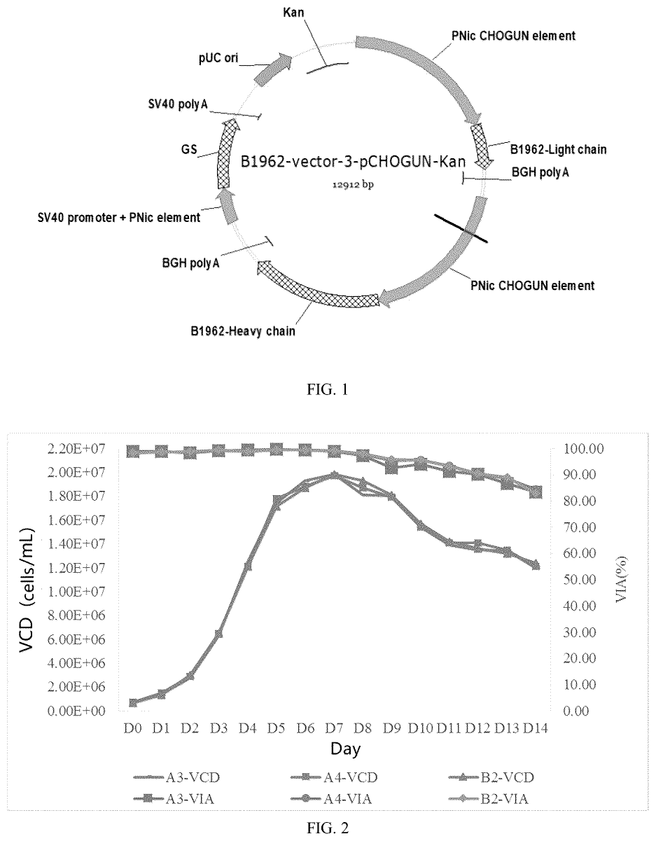

### DETAILED DESCRIPTION

To further elaborate on the technical means adopted and effects achieved in the present application, the present application is further described below in conjunction with examples and drawings. It is to be understood that the specific examples set forth below are intended to explain the present application and not to limit the present application.

Experiments without specific techniques or conditions specified in the examples are conducted according to techniques or conditions described in the literature in the art or product specifications. The reagents or instruments used herein without manufacturers specified are conventional products commercially available from proper channels.

### Example 1

In this example, a cell line expressing a bispecific antibody PD-L1/VEGF was constructed.

In this example, biogenic materials were Chinese hamster ovary (CHO) cells, and an expression vector B1962 vector was named B1962-vector-3-pCHOGUN-Kan. The plasmid contained an SV40 promoter for mediating high expression of a recombinant protein, a GS (a glutamine synthetase gene), SV40 polyA (a poly A tail signal for effectively terminating transcription of mRNA and enabling the mRNA to be polyadenylated), Kan (a Kana resistance gene for screening when E. coli was transformed), a pNic CHOGUN element and BGH polyA (a BGH polyadenylation signal). Used host cells were CHO cells, which were CHO-GS knockout expression systems. The CHO cells were purchased from Horizon Discovery Ltd. A GS-CHO-K1 working cell bank (MCB) with a generation of P10 and a GS-CHO-K1 WCB with a generation of P13 were established by Tasly Biopharmaceuticals Co., Ltd.

Target amino acid sequences (SEQ ID NO. 1 and SEQ ID NO. 2) were developed by AP Biosciences. On the basis of the amino acid sequences developed by AP Biosciences and on the premise that amino acid sequences of the PD-L1/VEGF bispecific antibody were not changed, the sequences of the PD-L1/VEGF bispecific antibody were optimized at a DNA level according to a codon preference of the host cells GS-CHO-K1. The target gene sequences of the antibody are shown in SEQ ID NO. 3 and SEQ ID NO. 4.

(a DNA sequence of a heavy chain of the bispecific antibody):
SEQ ID NO. 3
caggtgcagctggtgcagtccggcgccgaggtgaagaagcctggctcctccgtgaaggtgagctgtaaggcttccggcggcaccttca
ggaggtacagcatcagctgggtgaggcaggcccctggccagggactggagtggatgggggcatcatccctgtgttcggcgctgctaag
tacgcccagaagttccagggccgggtgaccatcaccgccgatgagttcaccagcaccgcctacatggagctgtcctccctgacctccg
aggataccgctgtgtattattgtgccctgtccggcgacagcgatgccttcgacatctggggccagggcacaatggttaccgtgtcctc
cgcttccaccaagggcccctccgtgttccccctggccccttcttccaagtccaccagcggcggcaccgccgctctgggatgtctggtg
aaggattacttccctgagcctgtgaccgtgagctggaatagcggcgctctgaccagcggcgtgcacaccttccctgctgtgctgcaga
gcagcggcctgtactccctgtcctccgtggtgaccgtgcccagctcctccctgggcacccagacctacatctgtaatgtgaatcacaa
gcccagcaataccaaggtggacaagaaggtggagcccaagagctgcgataagacccacacctgtcctccttgtcccgcccccgagctg
ctgggaggaccatctgtgttcctgttccctcccaagcctaaggataccctgatgatctccaggacccctgaggtgacctgtgtggtgg
tggatgtgagccacgaggaccccgaggtgaagttcaactggtacgtggacggcgtggaggtgcacaatgccaagaccaagcccaggga
ggagcagtacgcttccacctacagggtggtgtccgtgctgaccgtgctgcaccaggactggctgaatggcaaggagtataagtgcgct
gtgagcaataaggctctgcccgcccccatcgagaaaactattagtaaggccaagggccagcccagggagccccaggtgtataccctgc
ccccttcccgggaggagatgaccaagaaccaggtgtccctgacctgtctggtgaaaggcttctacccttccgacatcgctgtggagtg
ggagagcaacggccagcccgagaacaattataagaccacccctcccgtgctggacagcgatggctccttcttcctgtacagcaagctg
accgtggacaagtccaggtggcagcagggcaatgtgttcagctgctccgtgatgcacgaggctctgcacaaccactacacccagaaga
gcctgagcctgtcccccggcggcggaggaggatctggaggaggaggcagcggcggcggaggttctggagacaccggctcccccttcgt
ggagatgtactccgagatccctgagatcatccacatgaccgagggctccgagctggtgatcccctgtcgggtgaccagccccgatatc
accgtgaccctgaagaagttccctctggataccctgatccccgacggcaagaggatcatctgggatagcaggaagggcttcatcatct
ccgatgctacctataaggagatcggcctgctgacctgtgaggctaccgtgaatggccacctgtacaagaccaactacctgacccaccg
gcagaccaataccatcatcgacgtggtgctgagccctagccacggcatcgagctgtccgtgggcgagaagctggtgctggactgcacc
gccaggaccgagctgaatgtgggcatcgacttcaactgggagtaccctagcagcaagcaccagcacaagaagctggtgaatagggacc
tgaaaactcaatctggcagcgagatgaagaagttcctgagcaccctgaccatcgatggcgtgaccaggtccgatcagggcctgtacac
ctgtgctgcttcttccggcctgatgaccaagaaggactccaccttcgtgagggtgcacgagaag.
(a DNA sequence of a light chain of the bispecific antibody):
SEQ ID NO. 4
cagagcgtgctgacccagcccccttccgctagcggcacccctggacagagggtgaccatcagctgttccggcagcagcagcaacatcg
gctccaacaccgtgaactggtaccagcagctgcctggcaccgcccccaagctgctgatctatagcaacaaccagcggccctccggcgt
gcctgatcggttctccggctccaagtccggcacctccgcctccctggccatctccggtctgcagagcgaggatgaggccgactactac
tgcgctacctgggacctgagcctgaacgcttgggtggtgttcggcggcggcaccaagctgaccgtgctgggacagcctaaggctgctc
cctccgtgaccctgttccctcctagctccgaggagctgcaggctaataaggctaccctggtgtgcctgatctccgacttctatcccgg
cgccgtgaccgtggcttggaaggctgactccagccccgtgaaggccggagtggagaccaccaccccttccaagcagagcaacaataag
tacgctgccagcagctatctgagcctgacccccgagcagtggaagagccaccggagctatagctgccaggtgacccacgagggctcca
ccgtggagaaaactgttgctcccaccgagtgtagc.

The expression vector was introduced into the host cells GS-CHO-K1 through an electrotransfection method. In a subculture process, the host cells GS-CHO-K1 were screened by a medium that did not contain glutamine (Gln) but contained methionine sulfoximine (MSX) to obtain a stable cell population, and a series of screening such as a limited dilution method (0.45 cells/well, 96-well plate), monoclonal imaging (after the stable cell population was plated, the 96-well plate was centrifuged, photographed for the first time and subsequently photographed at 24 h, 48 h, 72 h and 168 h) and expression amount detection were performed to obtain a monoclonal cell line. The high-yielding monoclone was fed-batch cultured to obtain an optimal clone according to a growth state, a quality analysis (key quality attributes such as purity and activity), molecular characterization (an analysis of a relative molecular weight through a high-resolution mass spectrometry method, a coverage rate of peptide fragments, an analysis of a sequence at an N/C end through a mass spectrometry method and an analysis of a sequence at an N end through an Edman degradation method), genome level sequencing confirmation and a preliminary stability study. The optimal clone was named 131-35.

### Example 2

In this example, fed-batch culture was performed in shake flasks.

According to a determined process of fed-batch culture in the shake flasks, a confirmation experiment of the process of fed-batch culture at a shake flask stage was performed. A basal medium was Dynamis™ AGT™ Medium (containing 1.0 g/L Pluronic® F-68 BioChemica), and a feed medium was Cell Boost™ 7a and Cell Boost™ 7b. The cells were inoculated according to an inoculation density of 0.60×10⁶ cells/mL with a culture volume of 50 mL. Three parallels (numbered 35-12, 35-13 and 35-14) were inoculated in total. A process confirmation solution is shown in Table 1, and cell growth data and expression amounts of target proteins are shown in Table 2. After the samples (35-12, 35-13 and 35-14) were purified by Capto adhere, key quality attributes were detected. The results are shown in Table 3. The SEC-HPLC, CE-SDS and iCIEF data of the three shake flask samples are comparable.

TABLE 1

| Inoculation | Cell Boost ™ 7a (v/v) | | | | | Cell Density |
|---|---|---|---|---|---|---|
| Density | Day 3 | Day 4 | Day 5 | Day 6-End | | during Cooling |
| (cells/mL) | (D 3) | (D 4) | (D 5) | of culture | Cell Boost ™ 7b (v/v) | (cells/mL) |
| 0.60 × 10⁶ | 2.00% | 2.75% | 3.00% | 3.00% | D 3-D 5: 0.200% | 13.00 × 10⁶ |
| | | | | | D 6-D 10: 0.250% | |
| | | | | | D 11-End of culture: 0.225% | |

TABLE 2

| | Highest | Viability Rate | | |
|---|---|---|---|---|
| | Culture | at the End of | Period of | |
| | Density | Culture | Culture | Yield |
| No. | (cells/mL) | (%) | (days) | (g/L) |
| 35-12 | 21.50 × 10⁶ | 90.20 | 14 | 6.655 |
| 35-13 | 21.00 × 10⁶ | 87.08 | 14 | 6.617 |
| 35-14 | 20.90 × 10⁶ | 85.96 | 14 | 6.541 |

TABLE 3

| | SEC-HPLC | | R-CE-SDS | NR-CE-SDS | iCIEF | |
|---|---|---|---|---|---|---|
| No. | HMW (%) | Main Peak (%) | Purity (%) | Purity (%) | Isoelectric Point | Main Peak (%) |
| 35-12 | 2.4 | 97.6 | 98.1 | 95.9 | 7.6 | 58.8 |
| 35-13 | 1.9 | 98.1 | 98.3 | 95.9 | 7.6 | 64.9 |
| 35-14 | 1.9 | 98.1 | 98.0 | 95.9 | 7.6 | 59.4 |

### Example 3

In this example, cell culture was performed in 5 L bioreactors.

Three tubes of WCB cells were resuscitated to 250 mL shake flasks with a culture volume of 80 mL. The cells were cultured for 3 days and amplified to 1 L shake flasks at a density of 0.45×10⁶ cells/mL with a culture volume of 250 mL. The cells were cultured in the 1 L shake flasks for three days and amplified to 2 L shake flasks at a density of 0.55×10⁶ cells/mL with a culture volume of 600 mL. The cells were cultured in the 2 L shake flasks for three days with a viable cell density of >5.00×10⁶ cells/mL and a viability rate of >90.00%. The cells were inoculated in three 5 L reactors A3, A4 and B2. Media were Dynamis™ AGT™ Media (containing 1.0 g/L Pluronic® F-68 BioChemica). Inoculation densities of the three reactors A3, A4 and B2 were all 0.65×10⁶ cells/mL. Fed-batch was started on day 3 (D3), and cooling culture was started on D4. A fed-batch process of the fed-batch culture and a glucose addition amount are shown in Table 4. The culture was performed in the three reactors until day 4 (D4) (with a cell density of ≥12.00×10⁶ cells/mL) and cooled to 33° C. When a viability rate of the cell culture was lower than 70.00%, the culture was terminated. Key parameters of the reactors are shown in Table 5.

TABLE 4

| Cell Boost ™ 7a | | | | | Cell Boost ™ 7b | | | Glucose |
|---|---|---|---|---|---|---|---|---|
| D 3 | D 4 | D 5 | D 6 | D 7-end | D 3-D 5 | D 6-D 9 | D 10-end | Addition |
| 2.00% | 2.75% | 3.00% | 3.00% | 3.00% | 0.200% | 0.250% | 0.225% | start glucose |
| | | | | | | | | addition on |
| | | | | | | | | D 4 to 5 g/L |

TABLE 5

| | |
|---|---|
| pH (after inoculation) | 7.05 ± 0.25 |
| Temperature | 36.5° C. during inoculation |
| | culture to cell density of ≥12.00 × 10⁶ |
| | cells/mL and cool to 33.0° C. |
| Dissolved Oxygen (DO %) | 40.0% |
| Rotation Speed (rpm) | 200→220 (after inoculation, culture until |
| | day 3) |
| Surface Ventilation (1 pm) | 0-0.10 |
| Deep Ventilation (1 pm) | 0-0.45 |
| O₂ SPARGE | 0-0.45 |
| CO₂ SPARGE | Cascade to pH |

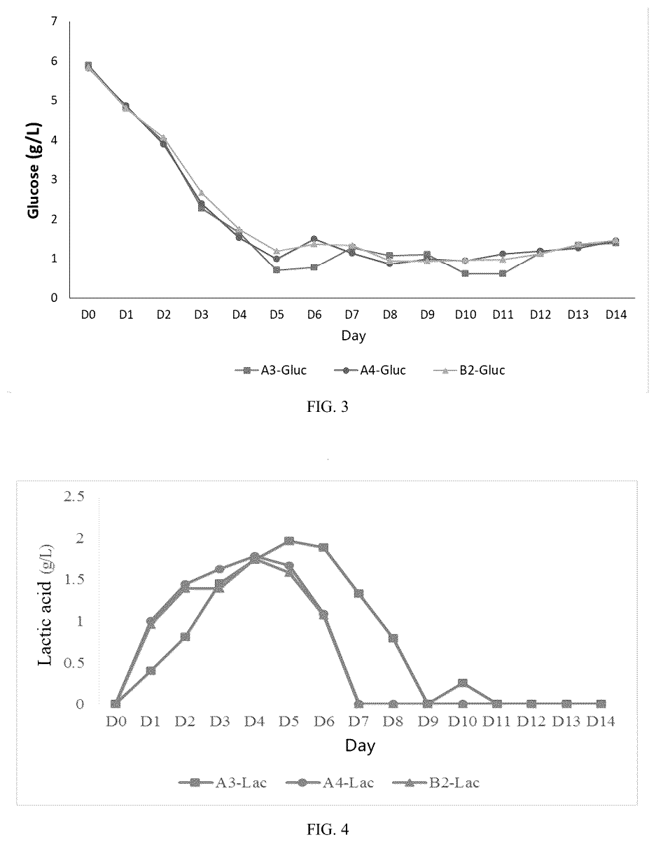

Cell densities, viability rates, culture days and expression amounts of target proteins of the three 5 L reactors from the inoculation to the end of culture are shown in Table 6, and growth curves of the cells are shown in FIG. 2. The cells in the A3, A4 and B2 reactors all reached a density peak of about 20.00×10⁶ cells/mL on day 7 (D7). In an entire process of fed-batch culture, the cell densities and the viability rates were normal, the densities and the viability rates of the three batches of parallel reactors were basically the same, and at the end of culture, the viability rates of the cells were all greater than 80.00%. Cell metabolism tracked by central control is shown in FIGS. 3 to 5. The detection values of glucose were relatively stable. At a later stage of fed-batch, glucose was maintained at 0.6-1.6 g/L. When the fed-batch culture was performed until D6, lactic acid started to decrease. From the later stage to the end of culture, contents of lactic acid were extremely low. In an entire fed-batch process, NH₄⁺ had a tendency to accumulate and increase, and an accumulation process was relatively smooth.

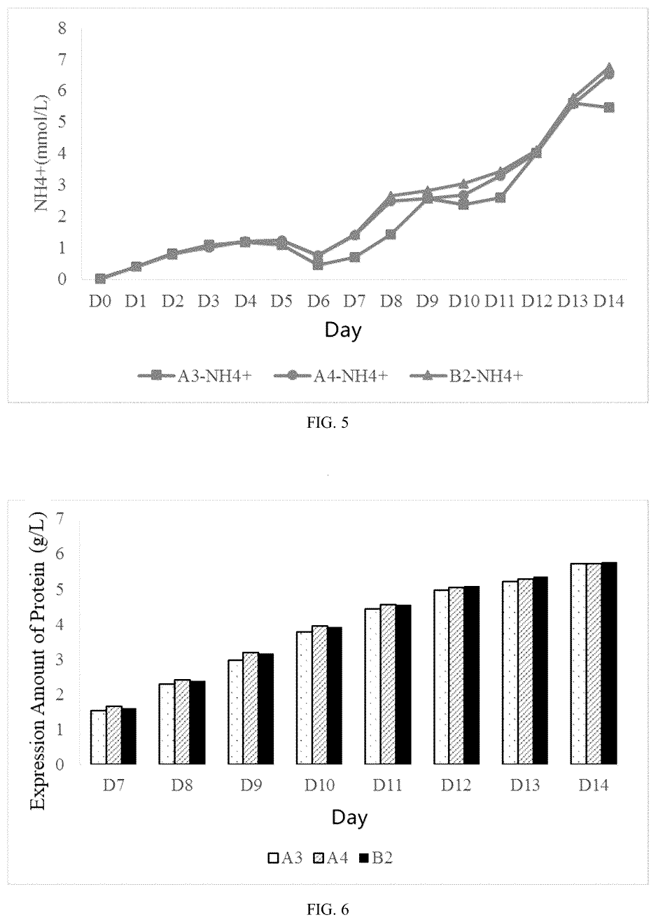

The expression amounts of the proteins of the three reactors are shown in FIG. 6, and polymer proportions in cell culture solutions are shown in Table 6. At the end of cell culture, the polymer proportions (HMW) of the three parallel reactors were all lower than 8.0%. After fermentation broths were affinity captured by MabSelect PrismA and purified by Capto adhere, SEC-HPLC, CE-SDS and iCIEF were detected. The results are shown in Table 7. There is little difference between the results of SEC-HPLC, CE-SDS and iCIEF in parallel batches of the three reactors, and consistency 5 between the batches is relatively good.

TABLE 6

| | Inoculation | Highest | Density at the | Viability Rate at | Period of | | Polymer |
|---|---|---|---|---|---|---|---|
| Tank | Density | Density | End of Culture | the End of Culture | Culture | Yield | Proportion |
| No. | (cells/mL) | (cells/mL) | (cells/mL) | (%) | (days) | (g/L) | (%) |
| A3 | 0.68 × 10⁶ | 19.80 × 10⁶ | 12.00 × 10⁶ | 83.39 | 14 | 5.759 | 6.7 |
| A4 | 0.69 × 10⁶ | 19.70 × 10⁶ | 12.10 × 10⁶ | 84.26 | 14 | 5.812 | 7.6 |
| B2 | 0.68 × 10⁶ | 19.80 × 10⁶ | 12.40 × 10⁶ | 82.83 | 14 | 5.830 | 7.6 |

TABLE 7

| | | | | | iCIEF | | ELISA Binding | |
|---|---|---|---|---|---|---|---|---|
| | SEC-HPLC | | CE-SDS | | | ICIEF | Activity | |
| Reactor | Polymer | Monomer | R-CE-SDS | NR-CE-SDS | Isoelectric | Main Peak | PD-L1 | VEGF |
| No. | (%) | (%) | (%) | (%) | Point | (%) | (%) | (%) |
| A3 | 0.8 | 99.2 | 97.8 | 96.6 | 7.7 | 51.3 | 113 | 106 |
| A4 | 0.8 | 99.2 | 96.0 | 97.0 | 7.7 | 52.4 | 93 | 89 |
| B2 | 0.8 | 99.2 | 96.1 | 97.3 | 7.7 | 52.7 | 95 | 96 |

### Example 4

In this example, cell culture was performed in 200 L bioreactors.

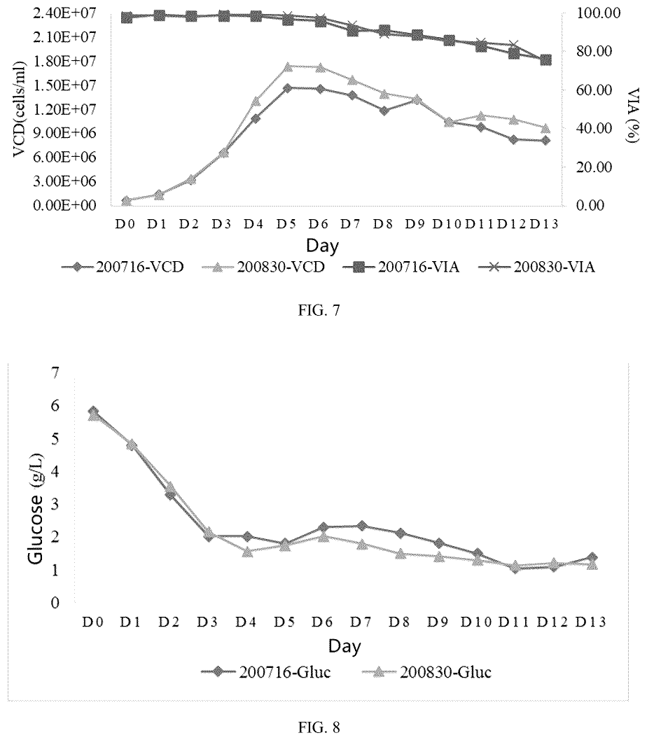

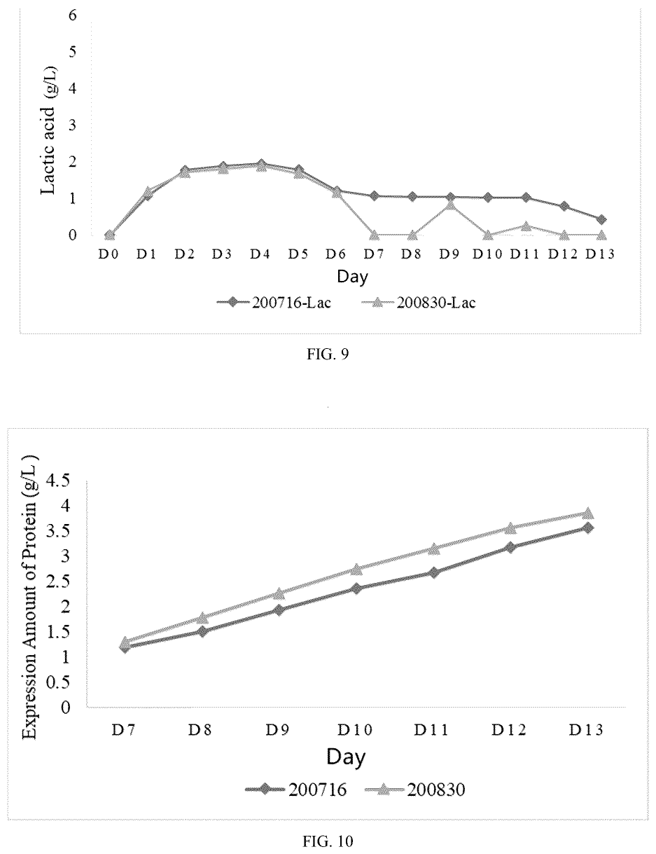

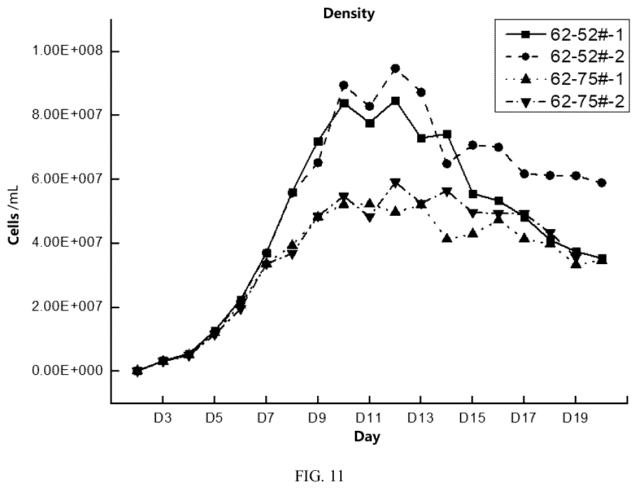

Under GMP conditions, two batches of cell culture (Lot Nos. 200716 and 200830) with a scale of 200 L were performed. Process parameters in each operation step were controlled in a process of cell culture. A temperature, CO₂, a rotation speed and a cell density were controlled at a shake flask stage, a temperature, DO, a pH, a rotation speed and a cell density were controlled in a process of Wave culture, and a temperature, DO, a pH, a rotation speed, a period of culture and a viability rate of cells were controlled in a process of 200 L culture. The culture results are shown in Table 8, cell growth at a 200 L cell culture stage is shown in FIG. 7, metabolic parameters of glucose at the 200 L cell culture stage are shown in FIGS. 8 and 9, and expression yields of target proteins are shown in FIG. 10. The results indicate that a culture process is stable and reliable with reproducibility and the cell growth, the metabolism and the expression amounts of the proteins are relatively consistent under conditions of the culture process. The expression amounts of the target proteins in culture solutions are 3.560 g/L and 3.845 g/L, respectively.

TABLE 8

| | | |
|---|---|---|
| Batch | 200716 | 200830 |
| Cell Line | WCB, Batch: 20200602 | WCB, Batch: 20200602 |
| Number of Times of | 4 | 4 |
| Amplification of Seed | | |
| Resuscitation | | |
| Manner of Seed | shake flask culture (250 mL→1 | shake flask culture (250 mL→1 |
| Amplification | L→2 L) + Wave culture | L→2 L) + Wave culture |
| Density of Shake Flask | resuscitation: 0.46 × 10⁶ | resuscitation: 0.54 × 10⁶ |
| Culture (cells/mL) | 250 mL-D3: 4.20 × 10⁶ | 250 mL-D3: 4.65 × 10⁶ |
| | 1 L-1-D3: 5.70 × 10⁶ | 1 L-1-D3: 6.16 × 10⁶ |
| | 1 L-2-D3: 5.41 × 10⁶ | 1 L-2-D3: 6.04 × 10⁶ |
| | 2 L-1-D3: 6.21 × 10⁶ | 2 L-1-D3: 6.04 × 10⁶ |
| | 2 L-2-D3: 6.67 × 10⁶ | 2 L-2-D3: 6.40 × 10⁶ |
| | 2 L-3-D3: 6.34 × 10⁶ | 2 L-3-D3: 6.19 × 10⁶ |
| | 2 L-4-D3: 6.34 × 10⁶ | 2 L-4-D3: 6.33 × 10⁶ |
| Wave Culture (cells/mL) | D3: 6.07 × 10⁶ | D3: 6.49 × 10⁶ |
| Initial Inoculation | 15 L | 15 L |
| Amount of 200 L Reactor | | |
| Cooling Density | 12.90 × 10⁶ | 13.00 × 10⁶ |
| (cells/mL) | | |
| Highest Density | 14.60 × 10⁶ | 17.30 × 10⁶ |
| (cells/mL) | | |
| Viability Rate at the End | 75.80% | 74.82% |
| of Culture | | |
| Culture Days | 13 days | 13 days |
| Expression Amount of | 3.560 | 3.845 |
| Protein on Day 13 (g/L) | | |

### Example 5

In this example, perfusion culture was performed.

With a culture scale of 50 mL TPP culture tube, perfusion media used were Eden-300S (BioEngine, the medium was named 52 # in this example) and a High-Intensity Perfusion CHO Medium (gibco, the medium was named 75 # in this example).

The 52 #perfusion medium was combined with feeds Eden-F400a (BioEngine) and Eden-F200b (BioEngine), that is, according to cell growth, Eden-F400a was supplemented to 2.5% to 12% of a culture volume, and Eden-F200b was supplemented to 10% of an additional volume of Eden-F400a. Glucose was controlled at about 10 g/L at an early stage of cell perfusion when the 52 #perfusion medium was used, glucose was controlled at 10-20 g/L at middle and later stages of perfusion, and glucose was controlled at 10-12 g/L in a cell perfusion process where the 75 #perfusion medium was used.

When a cell density of the 52 #medium reached 3×10⁷ cells/mL, a temperature was lowered to 33° C., and when a cell density of the 75 #medium reached 3.5×10⁷ cells/mL, a temperature was lowered to 33° C.

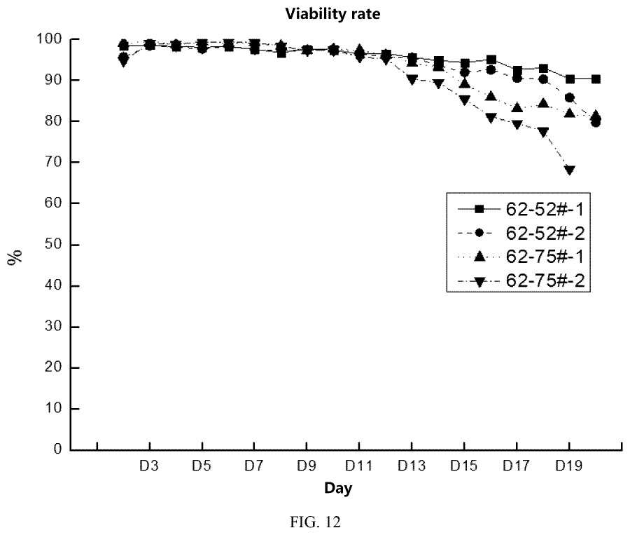

One culture tube of the 52 #medium had a cell density of 6.5×10⁷-9.0×10⁷ cells/mL, and a cell solution that was 10% of a culture volume was discharged every day; one culture tube of the 75 #medium had a cell density of 4.5×10⁷-6.0×10⁷ cells/mL, and a cell solution that was 10% of a culture volume was discharged every day. A cell culture cycle was 20 days. Densities and viability rates of perfusion cultured cells are shown in FIGS. 11 and 12. Cells in the 52 #medium had a relatively high density peak, which was about 8.0×10⁷-10.0×10⁷ cells/mL; cells in the 75 #medium had a density peak of about 5.0×10⁷-6.0×10⁷ cells/mL. Expression amounts of the perfusion culture are shown in Table 9. 75 # had an expression amount of >2 g/L every day at the middle and later stages of perfusion, and 52 # had a higher expression amount every day at the middle and later stages of perfusion, which was >3 g/L.

TABLE 9

| | Expression Amount (g/L) | | | | | |
|---|---|---|---|---|---|---|
| Cell- | day 9 | day 11 | day 13 | day 16 | day 18 | day 20 |
| Abbreviation | (D 9) | (D 11) | (D 13) | (D 16) | (D 18) | (D 20) |
| 62-52#-1 | 0.966 | 2.003 | 2.683 | 3.114 | 3.851 | 3.398 |
| 62-52#-2 | N/A | 2.184 | 2.852 | 3.032 | 3.005 | 2.977 |
| 62-75#-1 | 0.949 | 1.695 | 2.17 | 2.535 | 2.984 | 2.703 |
| 62-75#-2 | N/A | 1.869 | 2.483 | 3.004 | 3.275 | 2.543 |

### Example 6

In this example, purification was performed on the PD-L1/VEGF bispecific antibody.

On the basis of the development and confirmation of a 5 L small-scale purification process, a purification process of 200 L fermentation scale was amplified and studied, a purification process of a stock solution was established, and a purification process was determined, where affinity chromatography using a Mabselect PrisemA filler (CYTIVA Medical Group) was used as a basis, low-pH incubation (pH=3.5±0.1) was used for the first virus removal, anion chromatography using a Capto adhere composite filler (CYTIVA Medical Group) and cation-exchange chromatography using a Nuvia HRS filler (BIORAD) were used for fine purification, and filtration using a 1.0 m² Bio EX nanofiltration membrane (Asahi Kasei Corporation) was used for the third virus removal, and a tangential flow ultrafiltration membrane P2B050A25 (Merck Millipore) with a molecular weight cut-off of 50 kDa was used for the preparation of a concentrated stock solution. In a purification process, a microbial limit, an HCP residual, a DNA residual, an endotoxin, a content of an intermediate and purity were controlled and detected. Two batches of stock solutions were produced under the GMP conditions. After release inspection, the stock solutions both met quality standards. The process was stable and reliable, and consistency between the batches was relatively good. The batches were used for IND reporting.
1. The preparation of a solution in each step of a process of amplifying the purification is shown in Table 10.

TABLE 10

| | | Preparation |
|---|---|---|
| Buffer Name | Components and Conditions | Volume (L) |
| Buffer A | 20 mM PB, 150 mM NaCl, pH 7.2 ± 0.2, cond 18.0 ± 2.0 | 400 |
| | ms/cm | |
| Buffer B | 20 mM acetic acid-sodium acetate, 1M NaCl, pH | 200 |
| | 5.5 ± 0.2, cond 86.0 ± 2.0 ms/cm | |
| Buffer C | 20 mM acetic acid-sodium acetate, pH 5.5 ± 0.2, cond | 200 |
| | 1.5 ± 1.0 ms/cm | |
| Buffer D | 20 mM acetic acid-sodium acetate, pH 3.5 ± 0.2, cond | 200 |
| | 0.2 ± 0.2 ms/cm | |
| Buffer E | 20 mM acetic acid-sodium acetate, pH 5.9 ± 0.1, cond | 400 |
| | 1.5 ± 0.5 ms/cm | |
| Buffer F | 20 mM acetic acid-sodium acetate, pH 4.0 ± 0.1, cond | 200 |
| | 0.3 ± 0.2 ms/cm | |
| Buffer G | 20 mM acetic acid-sodium acetate, 100 mM Arg, pH | 200 |
| | 5.5 ± 0.1, cond 9.0 ± 1.0 ms/cm | |
| Buffer H | 190 mM Arg, pH 5.5 ± 0.1, cond 14.0 ± 1.0 ms/cm | 200 |
| Buffer I | 1M Arg, cond 43.0 ± 2.0 ms/cm | 7.5 |
| Buffer J | 10 mM acetic acid-sodium acetate, pH 5.0 ± 0.2, cond | 50 |
| | 0.5 ± 0.3 ms/cm | |
| Buffer K | 1M NaOH | 5 |
| Buffer L | 0.5M NaOH | 200 |
| Buffer M | 0.1M NaOH | 50 |
| Buffer N | 1M NaCl | 200 |
| Buffer O | 20 mM histidine, pH 6.0, cond | 200 |
| 3M | 3M hydrochloric acid | 1 |
| Hydrochloric | | |
| Acid | | |
| 20% Ethanol | 20% ethanol | 30 |

2. Operating parameters of each purification process are listed below according to process steps.

### (1) Depth Filtration and Clarification

A depth filter, which was specifically designed for the distribution of particle diameters of a pretreated feed liquid, was a clarification filter with a gradient density structure. The depth filtration and clarification were performed on fermented samples to remove large particulate matters and prepare for capture. Operating parameters of a process of amplifying the depth filtration are shown in Table 11. Two batches of experiments (numbered 200716 and 200830) were performed. Product purity and yields obtained through a process of depth filtration are shown in Table 12.

TABLE 11

| | Process | | | |
|---|---|---|---|---|
| Process Step | Parameter | Range | 200716 | 200830 |
| installation | manufacturer/lot | 3M/ | S232474301- | S239339301- |
| | number | E16E07A60 | 007/S232474301- | 50/S239339301-57 |
| | | SP02A | 008 | |
| equilibration | volume | 54.0 ± 5.0 L/m² | 170.0 L | 170.0 L |
| sample | sample | less than | 173.9 L | 172.5 L |
| loading | loading | 190 L/m² | | |
| | amount | | | |
| post-flush | volume | 10-20 L/m² | 45.0 L | 45.0 L |

TABLE 12

| | Lot No. | |
|---|---|---|
| Item | 200716 | 200830 |
| purity (%) | 50.8% | 50.7% |
| yield (%) | 95.9% | 89.6% |

### (2) Processes of Affinity Chromatography Capture and Low-pH Incubation for Viral Inactivation

Specific adsorption of target protein antibodies and Proteins A was used in the affinity capture chromatography to achieve an object of capturing target proteins. Operating parameters of a process of amplifying MabSelect PrismA capture are shown in Table 13, and product purity and yields are shown in Table 14.

TABLE 13

| | Process | Parameter | | |
|---|---|---|---|---|
| Process Step | Parameter | Range | 200716 | 200830 |
| preparation of | diameter | 300 mm | 300 mm | 300 mm |
| chromatography | column | 14.0 ± 1.0 cm | 13.5 cm | 13.5 cm |
| column | height | | | |
| | column | 9.9 ± 0.7 L | 9.5 L | 9.5 L |
| | volume | | | |
| | symmetry | 0.8-1.8 | 1.10 | 1.07 |
| | column | >2000 N/m | 3585 N/m | 3301 N/m |
| | efficiency | | | |
| | flow rate | period of | 114.5 L/h | 114.5 L/h |
| | | contact: 5 min | | |
| pre-CIP | flush with | ≥2.5 CV | 2.5 CV | 2.5 CV |
| | water for | | | |
| | injection | | | |
| | 0.5M | ≥30 min | 30 min | 30 min |
| | NaOH | | | |
| | flush with | ≥2.5 CV | 2.5 CV | 2.5 CV |
| | water for | | | |
| | injection | | | |
| pre-equilibrium | equilibrium | 5.0 ± 0.5 CV | 5.1 CV | 5.1 CV |
| | volume | | | |
| sample loading | sample | ≤37.0 g/L filler | 28.3/29.0 g/L | 28.5/28.8 g/L |
| | loading | | | |
| | amount | | | |
| post-equilibrium | equilibrium | 5.0 ± 0.5 CV | 5.1 CV | 5.1 CV |
| | volume | | | |
| pre-wash 1 | pre-wash | 5.0 ± 0.5 CV | 5.1 CV | 5.1 CV |
| | volume | | | |
| pre-wash 2 | pre-wash | 5.0 ± 0.5 CV | 5.1 CV | 5.1 CV |
| | volume | | | |
| elution | start | 500 ± 50 mAu | 500 mAu | 500 mAu |
| | collection | | | |
| | end | 500 ± 50 mAu | 500 mAu | 500 mAu |
| | collection | | | |
| incubation | pH | 3.5 ± 0.1 | 3.515/3.581 | 3.514/3.520 |
| | period | 60 ± 10 min | 66/60 min | 64/64 min |
| neutralization | pH | 5.0 ± 0.1 | 4.930/4.921 | 4.948/4.946 |
| post-CIP | water for | 2.5 ± 0.5 CV | 2.5 CV | 2.5 CV |
| | injection | | | |
| | 0.5M | ≥30 min | 30 min | 30 min |
| | NaOH | | | |

TABLE 14

| | Lot No. | |
|---|---|---|
| Item | 200716 | 200830 |
| purity (%) | 94.0%, 94.0% | 92.9%, 93.3% |
| yield (%) | 92.3%, 91.0% | 94.2%, 93.3% |

### (3) Capto Adhere Chromatography

Anions and hydrophobic ligands carried by Capto adhere were used for purifying specific adsorption of impurities such as DNA and host proteins. Residual DNA and HCP in the target proteins were removed. Neutralization, filtration and an Adhere flow-through process were performed on the captured samples, and flow-through solutions were collected. Operating parameters of amplifying a process of Capto adhere chromatography are shown in Table 15, and product purity and yields obtained through the chromatography are shown in Table 16.

TABLE 15

| | Process | | | |
|---|---|---|---|---|
| Process Step | Parameter | Range | 200716 | 200830 |
| preparation of | diameter | 300 mm | 300 mm | 300 mm |
| chromatography | column | 16.0 ± 1.0 cm | 16.5 cm | 16.5 cm |
| column | height | | | |
| | column | 11.3 ± 0.7 L | 11.7 L | 11.7 L |
| | volume | | | |
| | symmetry | 0.8-1.8 | 1.17 | 1.10 |
| | column | >2000 N/m | 3430 N/m | 3696 N/m |
| | efficiency | | | |
| | flow rate | period of | 116.6 L/h | 116.6 L/h |
| | | contact: 6 | | |
| | | min | | |
| pre-CIP | flush with | ≥2.0 CV | 2.5/2.1 CV | 2.1/2.1 CV |
| | water for | | | |
| | injection | | | |
| | 0.5M NaOH | ≥30 min | 30 min | 30 min |
| | flush with | ≥2.0 CV | 2.5/2.1 CV | 2.1/2.1 CV |
| | water for | | | |
| | injection | | | |
| | 1.0M NaCl | ≥1.5 CV | 1.5/1.5 CV | 1.5/1.5 CV |
| | flush with | ≥2.0 CV | 2.1/2.1 CV | 2.1/2.1 CV |
| | water for | | | |
| | injection | | | |
| pre-equilibrium | volume | 10.0 ± 0.5 CV | 10.0/10.0 CV | 10.0/10.0 |
| | | | | CV |
| sample loading | load | ≤30.0 g/L | 18.9/18.9 g/L | 19.6/19.4 |
| | | filler | | g/L |
| | pH | 5.9 ± 0.1 | 5.860 | 5.820 |
| collection | start | 500 ± 50 mAu | 500 mAu | 500 mAu |
| | collection | | | |
| | end | 500 ± 50 mAu | 500 mAu | 500 mAu |
| | collection | | | |
| post- | volume | 2.0 ± 0.5 CV | 2.1/2.1 CV | 2.1/2.1 CV |
| equilibrium | | | | |
| elution | volume | 2.0 ± 0.5 CV | 2.1/2.1 CV | 2.1/2.1 CV |
| post-CIP | water for | ≥2.0 CV | 2.1/2.1 CV | 2.1/2.1 CV |
| | injection | | | |
| | 0.5M NaOH | ≥30 min | 30 min | 30 min |

TABLE 16

| | Lot No. | |
|---|---|---|
| Item | 200716 | 200830 |
| purity (%) | 95.6%, 95.3% | 97.6%, 96.9% |
| yield (%) | 85.8%, 86.1% | 85.3%, 89.2% |

### (4) Cation-Exchange Chromatography

Characteristics of the cation-exchange chromatography were used for separating and purifying components of the target proteins in an adsorption-elution mode. Aggregates and fragmented impurities whose properties were similar to those of the target proteins were mainly removed, and some host protein residues and DNA residues were removed, thereby achieving a purification effect. Operating parameters of a process of Nuvia HR-S cation-exchange chromatography are shown in Table 17, and product purity and yields obtained through the chromatography are shown in Table 18.

TABLE 17

| | Process | Parameter | | |
|---|---|---|---|---|
| Process Step | Parameter | Range | 200716 | 210830 |
| preparation of | diameter | 300 mm | 300 mm | 300 mm |
| chromatography | column height | 17.0 ± 1.0 cm | 18.0 cm | 18.0 cm |
| column | column | 12 ± 0.7 L | 12.7 L | 12.7 L |
| | volume | | | |
| | symmetry | 0.8-1.8 | 0.84 | 0.92 |
| | column | >2000 N/m | 5058 N/m | 6152 N/m |
| | efficiency | | | |
| | flow rate | period of | 109.0 L/h | 109.0 L/h |
| | | contact: 7 min | | |
| pre-CIP | flush with | ≥2.0 CV | 2.0 CV | 2.0 CV |
| | water for | | | |
| | injection | | | |
| | 0.5M NaOH | ≥30 min | 40 min | 37 min |
| | flush with | ≥2.0 CV | 2.0 CV | 2.0 CV |
| | water for | | | |
| | injection | | | |
| | 1.0M NaCl | ≥1.5 CV | 1.5 CV | 1.5 CV |
| | regeneration | | | |
| | flush with | ≥2.0 CV | 2.0 CV | 2.0 CV |
| | water for | | | |
| | injection | | | |
| pre-equilibrium | volume | 5.0 ± 0.5 CV | 5.0 CV | 5.0 CV |
| sample loading | sample | ≤35.0 g/L filler | 30.0 g/L | 31.4 g/L |
| | loading | | | |
| | amount | | | |
| | pH after | 5.5 ± 0.1 | 5.499 | 5.480 |
| | sample | | | |
| | adjustment | | | |
| post- | volume | 5.0 ± 0.5 CV | 5.0 CV | 5.0 CV |
| equilibrium | | | | |
| elution | start collection | 1000 ± 100 mAu | 1000 mAu | 1000 mAu |
| | end collection | 1000 ± 100 mAu | 1000 mAu | 1000 mAu |
| post-CIP | flush with | ≥2.0 CV | 2.0 CV | 2.0 CV |
| | water for | | | |
| | injection | | | |
| | 0.5M NaOH | ≥30 min | 30 min | 30 min |
| | 0.1M NaOH | >1.0 CV | 1.2 CV | 1.6 CV |
| | storage | | | |

TABLE 18

| | Lot No. | |
|---|---|---|
| Item | 200716 | 200830 |
| purity (%) | 99.0% | 99.0% |
| yield (%) | 91.7% | 93.9% |

### (5) Nanofiltration for Virus Removal

Operating parameters of a process of amplifying the nanofiltration are shown in Table 19, and a monitoring table of a water flux of the nanofiltration membrane is shown in Table 20.

TABLE 19

| | Process | | | |
|---|---|---|---|---|
| Process Step | Parameter | Range | 200716 | 200830 |
| installation | pre-filtration | lot | 85000400 | 919007003- |
| | membrane | number/membrane | 3-3/0.2 | 2/0.2 m² |
| | (Sartorius | area | m² | |
| | AG) | | 85000400 | 919007003- |
| | | | 3-10/0.2 | 7/0.2 m² |
| | | | m² | |
| | nanofiltration | lot | F1909242 | F2001272124/ |
| | membrane | number/membrane | 125/1 m² | 1 m² |
| | (Asahi Kasei | area | | |
| | Corporation) | | | |
| equilibration | volume | 25.0 ± 0.5 L/m² | 25.0 L/m² | 25.0 L/m² |
| post-flush | volume | 5.0 ± 0.2 L/m² | 5.0 L/m² | 5.0 L/m² |

TABLE 20
Curve of Load of Nanofiltration Membrane

| | | | Permeate | |
|---|---|---|---|---|
| Period of | Permeate | Filtration | Flow | Process |
| Filtration/min | Mass/kg | Pressure/bar | Rate/LMH | Discription |
| 0 | 0 | 0.9 | 0.0 | start |
| | | | | nanofiltration |
| | | | | of sample |
| 10 | 2.5 | 1.9 | 15.0 | nanofiltration |
| 20 | 4.2 | 1.9 | 10.2 | nanofiltration |
| 30 | 5.7 | 2.2 | 9.0 | nanofiltration |
| 40 | 7.1 | 2.2 | 8.3 | nanofiltration |
| 50 | 8.6 | 2.3 | 9.0 | nanofiltration |
| 60 | 9.9 | 2.3 | 7.8 | nanofiltration |
| 70 | 11.3 | 2.3 | 8.3 | nanofiltration |
| 80 | 12.7 | 2.3 | 8.3 | nanofiltration |
| 90 | 14.1 | 2.3 | 8.3 | nanofiltration |
| 100 | 15.5 | 2.3 | 8.3 | nanofiltration |
| 110 | 16.9 | 2.3 | 8.3 | nanofiltration |
| 120 | 18.3 | 2.3 | 8.3 | nanofiltration |
| 130 | 19.8 | 2.3 | 8.3 | nanofiltration |
| 140 | 21.2 | 2.3 | 8.3 | nanofiltration |
| 150 | 22.7 | 2.3 | 8.3 | nanofiltration |
| 160 | 24.2 | 2.3 | 9.0 | nanofiltration |
| 170 | 25.7 | 2.3 | 9.0 | nanofiltration |
| 180 | 27.2 | 2.3 | 9.0 | end |
| | | | | nanofiltration |
| 188 | 28.4 | 2.3 | 9.0 | flush with |
| | | | | buffer |
| 200 | 31.7 | 2.3-0.5 | 9.0 | end flush with |
| | | | | buffer |

### (6) Preparation of Stock Solutions

The preparation of the antibody stock solutions was performed on products obtained through the nanofiltration. Operating parameters of a process of amplifying the preparation of the antibody stock solutions are shown in Table 21, and product purity and yields are shown in Table 22.

TABLE 21

| Process Step | Process Parameter | Range | 200716 | 200830 |
|---|---|---|---|---|
| installation | membrane | Merck | Merck | Merck |
| of | manufacturer/model/ | Millipore | Millipore/ | Millipore/ |
| membrane | membrane area | 50 kDa | 50 kDa/3.0 m² | 50 kDa/3.0 m² |
| | integrity before use | ≤36 cc/min/m² | pass | pass |
| | water flux before use | ≥6.0 L/m²/h/psi | 10.0 L/m²/h/psi | 9.5 L/m²/h/psi |
| pre-clean | 0.5M NaOH cycle | volume | 5.0 L | 5.0 L |
| | 0.5M NaOH cycle | period ≥ 1.0 h | 1.8 h | 1.5 h |
| | water for injection | volume | 30.0 L | 30.0 L |
| equilibrium | buffer volume | ≥5.0 L/m² | 8.3 L/m² | 8.3 L/m² |
| and sample | | | | |
| loading | load | ≤500 g/m² | 100 g/m² | 106 g/m² |
| buffer | multiple of buffer | 10.0 DV | 10.0 DV | 10.0 DV |
| replacement | replacement | | | |
| sterile | concentration | ≥30.0 g/L | 41.3 g/L | 40.9 g/L |
| filtration | (mg/mL) | | | |
| clean of | integrity of | ≤117 cc/min | pass | pass |
| membrane | membrane | | | |
| after use | 0.5M NaOH period | ≥30 min | 30 min | 30 min |
| | of cycle | | | |

TABLE 22

| | Lot No. | |
|---|---|---|
| Item | 200716 | 200830 |
| purity (%) | 99.0% | 99.1% |
| yield (%) | 100.0% | 100% |

It can be seen that controlling the purification process including an eluent of the affinity chromatography and the pH and filler of the cation chromatography can effectively remove multimers and significantly improve the yield and product purity of the bispecific antibody.

### Example 7

Compared with Example 3, Example 7 differs only in that the culture temperature was 31° C., and the others were the same as those of Example 3.

### Example 8

Compared with Example 3, Example 8 differs only in that the culture temperature was 37° C., and the others were the same as those of Example 3.

### Example 9

Compared with Example 3, Example 9 differs only in that the culture pH was 6.8, and the others were the same as those of Example 3.

### Example 10

Compared with Example 3, Example 10 differs only in that the culture pH was 7.3, and the others were the same as those of Example 3.

### Comparative Example 1

Compared with Example 3, Comparative Example 1 differs only in that the media Dynamis™ AGT™ Media were replaced with an equal amount of ActiPro™ media, and the others were the same as those of Example 3. Growth speeds of cells become slower, which finally affects density peaks of the cells, resulting in reduced yields.

### Comparative Example 2

Compared with Example 3, Comparative Example 2 differs only in that the media Dynamis™ AGT™ Media were replaced with an equal amount of ExpiCHO Stable Production Media, and the others were the same as those of Example 3. Growth speeds of cells become slower, which finally affects density peaks of the cells, resulting in reduced yields.

### Comparative Example 3

Compared with Example 3, Comparative Example 3 differs only in that the media Dynamis™ AGT™ Media were replaced with an equal amount of CD FortiCHO Media, and the others were the same as those of Example 3. Growth speeds of cells become slower, which finally affects density peaks of the cells, resulting in reduced yields.

### Comparative Example 4

Compared with Example 3, Comparative Example 4 differs only in that the culture temperature was 25° C., and the others were the same as those of Example 3. The relatively low culture temperature causes slow cell production, which finally affects density peaks of cells, resulting in reduced yields.

### Comparative Example 5

Compared with Example 3, Comparative Example 5 differs only in that the culture temperature was 40° C., and the others were the same as those of Example 3. The high temperature is detrimental to cell culture. Cells suffer from certain damage, and unstable degradation of products is caused.

### Comparative Example 6

Compared with Example 3, Comparative Example 6 differs only in that the culture pH was 5.5, and the others were the same as those of Example 3. Product proteins are relatively sensitive to pH. The relatively low pH causes the degradation of products, resulting in the loss of yields.

### Comparative Example 7

Compared with Example 3, Comparative Example 7 differs only in that the culture pH was 8.1, and the others were the same as those of Example 3. The excessively high pH has a certain inhibitory effect on cell growth, affects density peaks of cells and finally affects yields. Moreover, the high pH causes increased basic peaks of antibodies, which has a certain effect on the quality of products.

### Comparative Example 8

Compared with Example 3, Comparative Example 8 differs only in that the culture dissolved oxygen (OD) was 5%, and the others were the same as those of Example 3. The excessively low DO changes the metabolism of cells, increases proportions of lactic acid produced from glucose, significantly reduces effective utilizations of media, reduces expression levels of cell proteins and even causes gradual cell apoptosis due to hypoxia.

Compared Example 3 with Examples 7 to 9 and Comparative Examples 1 to 8, it can be seen that the yields of the bispecific antibodies in Examples 7 to 9 can also reach more than 5 g/L, while the yields of the bispecific antibodies in Comparative Examples 1 to 7 are all significantly reduced, indicating that the yield of the bispecific antibody is affected by multiple factors and is very sensitive to changes in all the factors. In the present application, all the influencing factors are comprehensively analyzed, systematically controlled and cooperated to function, thereby significantly improve the yield of the bispecific antibody.

To conclude, in the present application, various influencing factors in the fermentation culture, the separation and the purification are comprehensively analyzed. Through effective control, all the factors can be effectively cooperated, thereby efficiently producing the high-purity bispecific antibody whose daily yield obtained through the perfusion production can reach more than 3 g/L. After the purification, the SEC-HPLC purity of the bispecific antibody can reach more than 90%. Moreover, the production process is amplified and verified. The process is stable, reliable and low-cost, and compared with an existing production method, a great breakthrough is achieved, which is of great significance for the widespread clinical application of the bispecific antibody.

The applicant has stated that although the detailed method of the present application is described through the examples described above, the present application is not limited to the detailed method described above, which means that the implementation of the present application does not necessarily depend on the detailed method described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. A production method for a bispecific antibody, comprising the following steps:
(1) constructing cells for producing the bispecific antibody and screening; and
(2) culturing cells obtained through the screening in step (1) to obtain a culture solution, and separating and purifying to obtain the bispecific antibody;
wherein the cells comprise mammalian cells; and
a manner of the culture comprises fed-batch culture or perfusion culture;
wherein a medium for the fed-batch culture comprises a basal medium and a feed medium,
wherein the basal medium comprises a Dynamis™ AGT™ Medium, the feed medium comprises Cell Boost™ 7a and Cell Boost™ 7b, a temperature of the fed-batch culture is 31° C.-37° C., a pH of the fed-batch culture is 6.8-7.3, and dissolved oxygen of the fed-batch culture is more than 10%; and
a medium for the perfusion culture comprises a basal medium and a feed medium, wherein the basal medium comprises an Eden-300S Medium and a High-Intensity Perfusion CHO Medium, a temperature of the perfusion culture is 31° C.-37° C., a pH of the perfusion culture is 6.8-7.3, and dissolved oxygen of the perfusion culture is more than 10%.

2. The production method for a bispecific antibody according to claim 1, wherein the bispecific antibody comprises a PD-L1/VEGF bispecific antibody;
preferably, an amino acid sequence of the PD-L1/VEGF bispecific antibody comprises sequences shown in SEQ ID NO. 1 and SEQ ID NO. 2;
preferably, the mammalian cells comprise HEK 293 cells or Chinese hamster ovary cells, preferably the Chinese hamster ovary cells; and
preferably, the basal medium for the fed-batch culture contains Pluronic® F-68 BioChemica.

3. The production method for a bispecific antibody according to claim 1, wherein a fed-batch proportion of Cell Boost™ 7a is 2% to 3%, and a fed-batch proportion of Cell Boost™ 7b is 0.2% to 2.5%;
preferably, a seeding density of the fed-batch culture is not less than 0.15×10⁶ cells/mL;
preferably, the fed-batch culture further comprises adding glucose;
preferably, a fed-batch amount of glucose is 1.0-10.0 g/L; and
preferably, the feed medium for the perfusion culture comprises Eden-F400a and Eden-F200b.

4. The production method for a bispecific antibody according to claim 1, wherein the separating and purifying in step (2) comprise the following steps:
(1′) performing depth filtration on the culture solution to obtain a clear solution;
(2′) performing affinity chromatography on the clear solution;
(3′) adjusting a pH of a product obtained through the affinity chromatography, and incubating the product;
(4′) adjusting a pH of a product obtained through the incubation, and performing depth filtration on the product;
(5′) performing anion-exchange chromatography on a product obtained through the depth filtration;
(6′) performing cation-exchange chromatography on a product obtained through the anion-exchange chromatography; and
(7′) performing nanofiltration on a product obtained through the cation-exchange chromatography.

5. The production method for a bispecific antibody according to claim 4, wherein a filter element of a filter for the depth filtration in step (1′) comprises a Zeta Plus EZP filter element E16E07A60SP02A.

6. The production method for a bispecific antibody according to claim 4, wherein an elution buffer for the affinity chromatography in step (2′) comprises acetic acid and sodium acetate;
preferably, a filler of a chromatography column for the affinity chromatography in step (2′) comprises MabSelect PrismA;
preferably, the pH in step (3′) is 3-4; and
preferably, the incubation in step (3′) is performed for 50-70 min at a temperature of 18° C.-26° C.

7. The production method for a bispecific antibody according to claim 4, wherein a filler of a chromatography column for the anion-exchange chromatography in step (5′) comprises Capto adhere;
preferably, a load of Capto adhere is set to ≤30 g/L; and
preferably, the anion-exchange chromatography in step (5′) is performed at a pH of 5.8-6.0.

8. The production method for a bispecific antibody according to claim 4, wherein a filler of a chromatography column for the cation-exchange chromatography in step (6′) comprises Ceramic CM and/or Nuvia HR-S;
preferably, an equilibration buffer for the cation-exchange chromatography in step (6′) comprises acetic acid and sodium acetate;
preferably, the cation-exchange chromatography in step (6′) is performed at a pH of 5.4-5.6;
preferably, an eluent for the cation-exchange chromatography in step (6′) comprises arginine; and
preferably, a concentration of arginine in the eluent is 0.18-0.20 mol/L.

9. The production method for a bispecific antibody according to claim 4, wherein the separation and the purification further comprise steps of preparing an antibody stock solution;
preferably, a preparation method for the antibody stock solution comprises:
performing ultrafiltration on the product obtained through the cation-exchange chromatography, and performing filtration on a product obtained through the ultrafiltration by using a sterile filtration membrane to obtain the antibody stock solution.

10. The production method for a bispecific antibody according to claim 1, wherein the method comprises the following steps:
(1) constructing Chinese hamster ovary cells for producing the bispecific antibody, and screening;
(2) culturing cells obtained through the screening in step (1) to obtain a culture solution;
(3) performing depth filtration on the culture solution by using a Zeta Plus EZP filter element E16E07A60SP02A to obtain a clear solution;
(4) filling a chromatography column with MabSelect PrismA, performing affinity chromatography on the clear solution, and eluting the clear solution with an elution buffer containing acetic acid and sodium acetate;
(5) adjusting a pH of a product obtained through the affinity chromatography to 3.5±0.1, and incubating the product for 50-70 min at 18° C.-26° C.;
(6) adjusting a pH of a product obtained through the incubation to 5.4-5.6, and performing depth filtration on the product;
(7) filling a chromatography column with Capto adhere, equilibrating the column with an equilibration buffer containing acetic acid and sodium acetate, and performing anion-exchange chromatography on a product obtained through the depth filtration;
(8) filling a chromatography column with Ceramic CM and/or Nuvia HR-S, equilibrating the column with an equilibration buffer containing acetic acid and sodium acetate, and performing cation-exchange chromatography on a product obtained through the anion-exchange chromatography;
(9) performing nanofiltration on a product obtained through the cation-exchange chromatography; and
(10) performing ultrafiltration on a product obtained through the nanofiltration, and performing filtration on a product obtained through the ultrafiltration by using a sterile filtration membrane to obtain the antibody stock solution;
wherein a manner of the culture comprises fed-batch culture or perfusion culture;
wherein a medium for the fed-batch culture comprises a basal medium and a feed medium,
wherein the basal medium comprises a Dynamis™ AGT™ Medium, the feed medium comprises Cell Boost™ 7a and Cell Boost™ 7b, a temperature of the fed-batch culture is 31-37° C., a pH of the fed-batch culture is 6.8-7.3, and dissolved oxygen of the fed-batch culture is more than 10%; and
a medium for the perfusion culture comprises a basal medium and a feed medium, wherein the basal medium comprises an Eden-300S Medium and a High-Intensity Perfusion CHO Medium, a temperature of the perfusion culture is 31° C.-37° C., a pH of the perfusion culture is 6.8-7.3, and dissolved oxygen of the perfusion culture is more than 10%.
